(19) United States
(12) Patent Application Publication
(10) Pub. No.: US 2020/0205712 A1
(43) Pub. Date: Jul. 2, 2020

(54) ASSESSMENT OF RISK FOR MAJOR DEPRESSIVE DISORDER FROM HUMAN ELECTROENCEPHALOGRAPHY USING MACHINE LEARNED MODEL

(71) Applicant: X Development LLC, Mountain View, CA (US)

(72) Inventors: Sarah Ann Laszlo, Mountain View, CA (US); Gabriella Levine, San Francisco, CA (US); Georgios Evangelopoulos, Venice, CA (US)

(21) Appl. No.: 16/284,607

(22) Filed: Feb. 25, 2019

(30) Foreign Application Priority Data
Dec. 28, 2018 (GR) 20180100569

(51) Int. Cl. A61B 5/16; A61B 5/04; A61B 5/0484; A61B 5/00; A61B 5/0482; A61B 5/048

(52) U.S. Cl. CPC A61B 5/165; A61B 5/04012; A61B 5/04842; A61B 5/7267; A61B 5/0022; A61B 5/743; A61B 5/048; A61B 5/0006; A61B 5/0482

(57) ABSTRACT

Methods, systems, and apparatus, including computer programs encoded on a computer storage medium, for presenting a human participant with information known to stimulate a person's neural reward system. Receiving an EEG signal from a sensor coupled to the human participant in response to presenting the participant with the information, the EEG signal being associated with the participant's neural reward system. Contemporaneously with receiving the EEG signal, receiving contextual information related to the information presented to the human participant. Processing the EEG signal and the contextual information in real time using a machine learning model trained to associate depression in the person with EEG signals associated with the person's neural reward system and the presented information. Diagnosing whether the participant is experiencing depression based on an output of the machine learning model.

### CROSS-REFERENCE TO RELATED APPLICATION

This application claims priority to Greek Patent Application No. 20180100569, filed on Dec. 28, 2018, entitled “ASSESSMENT OF RISK FOR MAJOR DEPRESSIVE DISORDER FROM HUMAN ELECTROENCEPHALOGRAPHY USING MACHINE LEARNED MODEL,” the entirety of which is hereby incorporated by reference.

### FIELD

This specification relates generally to electroencephalogram (EEG) signal processing and analysis, and more specifically to systems and methods for diagnostic analysis of EEG signals to assess a participant's risk of Major Depressive Disorder (MDD) using a machine learned model.

### BACKGROUND

An electroencephalogram (EEG) is a measurement that detects electrical activity in a person's brain. EEG measures the electrical activity of large, synchronously firing populations of neurons in the brain with electrodes placed on the scalp.

EEG researchers have investigated brain activity using the event-related potential (ERP) technique, in which a large number of experimental trials are time-locked and then averaged together, allowing the investigator to probe sensory, perceptual, and cognitive processing with millisecond precision. However, such EEG experiments are typically administered in a laboratory environment by one or more trained technicians. EEG administration often involves careful application of multiple wet sensor electrodes to a person's scalp, acquiring EEG signals using specialized and complex equipment, and offline EEG signal analysis by a trained individual.

### SUMMARY

Machine learning techniques can be used to determine a participant's risk to suffer from mood disorders, such as MDD. The assessment can be applied, automatically, upon measurement of EEG signals from the participant.

In certain aspects, the disclosure features a system that operates by measuring human brain activity in response to a task which probes the participant's neural reward system, such as a reward task. Poor functioning of the neural reward system (as is known to be present in MDD, for example) is detected by a ML classifier. The extent of malfunction is compared to an existing database of healthy and depressed brain data. Based on the extent of malfunction, the participant can be diagnosed as either healthy or depressed. In some embodiments, a confidence interval can be placed on the likelihood that the user is suffering from or at risk for depression.

In general, innovative aspects of the subject matter described in this specification can be embodied in methods that include the actions of presenting a human participant with information known to stimulate a person's neural reward system. Receiving an EEG signal from a sensor coupled to the human participant in response to presenting the participant with the information, the EEG signal being associated with the participant's neural reward system. Contemporaneously with receiving the EEG signal, receiving contextual information related to the information presented to the human participant. Processing the EEG signal and the contextual information in real time using a machine learning model trained to associate depression in the person with EEG signals associated with the person's neural reward system and the presented information. Diagnosing whether the participant is experiencing depression based on an output of the machine learning model. Other implementations of this aspect include corresponding systems, apparatus, and computer programs, configured to perform the actions of the methods, encoded on computer storage devices.

These and other implementations can each optionally include one or more of the following features.

In some implementations, presenting a human participant with information known to stimulate a person's neural reward system includes: receiving an EEG signal from a sensor coupled to the human participant in response to presenting the participant with the information, the EEG signal being associated with the participant's neural reward system; contemporaneously with receiving the EEG signal, receiving contextual information related to the information presented to the human participant; processing the EEG signal and the contextual information in real time using a machine learning model trained to associate depression in the person with EEG signals associated with the person's neural reward system and the presented information; and diagnosing whether the participant is experiencing depression based on an output of the machine learning model.

In some implementations, the information known to stimulate the person's neural reward system is associated with a reward task.

In some implementations, the reward task includes two outcomes, a first outcome that corresponds to a win for the participant and a second outcome that corresponds to a loss for the participant, where the contextual information includes information about the outcome of the reward task.

In some implementations, the information known to stimulate a person's neural reward system includes presenting to the participant a graphical image of two objects, each object concealing an outcome that includes either a winning outcome or a losing outcome. Some implementations include prompting the participant to select one of the two objects, where the contextual information includes the outcome concealed by the selected one of the objects.

In some implementations, processing the EEG signals includes extracting one or more parameters characteristic of the EEG signals for inputting into the machine learning model. In some implementations, prior to extracting the one or more parameters, processing the EEG signals includes filtering the EEG signals.

In some implementations, in order to determine a depression diagnosis, the machine learning model identifies a strong response in a loss theta region of the EEG signals associated with the person's neural reward system.

In some implementations, generating an output associated with the determination includes providing, for display on a participant interface, a graphical representation that depicts the participant's EEG signals with that of a healthy individual and a depressed individual. In some implementations, the participant interface includes at least one visualization of the participant's EEG signals. In some implementations, the visualization can be a waveform of the signal, a heat map, or a time frequency representation.

In some implementations, the contextual information includes at least one of: the participant's location, a temperature of the participant's surroundings, an outcome that affects the participant's reward-related positivity, available computing devices, activities occurring on available computing devices, information from wearables, information from cameras, information from smart home devices, a current time, and current weather.

In some implementations, the machine learning model includes a neural network or other machine learning architecture.

In some implementations, the machine learning model has a mapping function that maps a time series or frequency spectrum of values corresponding to EEG signals to a classification based on labeled training data and the contextual information.

In some implementations, the machine learning model is trained using training data from healthy and depressed individuals, where the data is labeled for a depression diagnosis and associated with specific contextual information. In some implementations, the training data is labeled using depression diagnosis labels prior to training the model on the data.

In some implementations, prior to processing the EEG signals and the contextual information in real time using a machine learning model, pre-processing the EEG signals using bandpass filtering, linear de-trending, and/or another machine learning model.

Some implementations include prior to displaying the output through a participant interface on a device for a medical professional, sending the generated output to the device medical professional using a cloud service.

Therefore, without requiring a subjective diagnosis from a human doctor (e.g., a psychiatrist or mental health professional), the depression diagnosis system can measure quantitative differences between participants and, after training, provide classifications of EEG signals with respect to whether they came from a depressed, non-depressed, or at-risk individual.

Among other advantages, the disclosed technology can be used to accurately and rapidly diagnose whether a participant is suffering from Major Depressive Disorder (MDD). For example, systems can provide real-time EEG analysis to determine (e.g., diagnose) whether a participant is suffering from MDD. Conventional depression diagnosis typically involves subjective analysis by a trained medical professional. The disclosed EEG systems can capture quantitative differences between labeled EEG signals and assign a participant's EEG signals to the labeled category associated with depression (diagnosis) using machine learning techniques.

The details of one or more embodiments of the participant matter of this specification are set forth in the accompanying drawings and the description below. Other features, aspects, and advantages of the participant matter will become apparent from the description, the drawings, and the claims.

### BRIEF DESCRIPTION OF THE DRAWINGS

FIG. 1 is a schematic diagram of an embodiment of an EEG system used to diagnose depression in a participant.

FIG. 2 is a flowchart showing aspects of the operation of the EEG system shown in FIG. 1.

FIG. 3 is another flowchart showing aspects of the operation of the EEG system shown in FIG. 1.

FIGS. 4A and 4B are plots comparing averaged ERP signals from participants who are suffering from MDD (FIG. 4A) and other participants who are healthy (FIG. 4B).

FIG. 5 is a flowchart showing other aspects of the operation of the EEG system shown in FIG. 1.

FIGS. 6A and 6B are plots illustrating the difference between win and loss trials of a reward task for a depressed participant (FIG. 6A) and a healthy participant (FIG. 6B).

FIGS. 6C and 6D are plots illustrating the difference between trials of a visual image task for a depressed participant (FIG. 6C) and a healthy participant (FIG. 6D).

FIGS. 6E-6H are plots that illustrate individual EEG trials used to generate the plots in FIGS. 6C and 6D.

FIGS. 7A to 7D are EEG spectrograms showing brain activity associated with a positive outcome (FIG. 7A) and a negative outcome (FIG. 7B) in a healthy participant, and showing brain activity associated with a positive outcome (FIG. 7C) and a negative outcome (FIG. 7D) in a depressed participant.

FIG. 8 is a schematic diagram of a data processing apparatus that can be incorporated into an EEG system.

Like reference numbers and designations in the various drawings indicate like elements.

### DETAILED DESCRIPTION

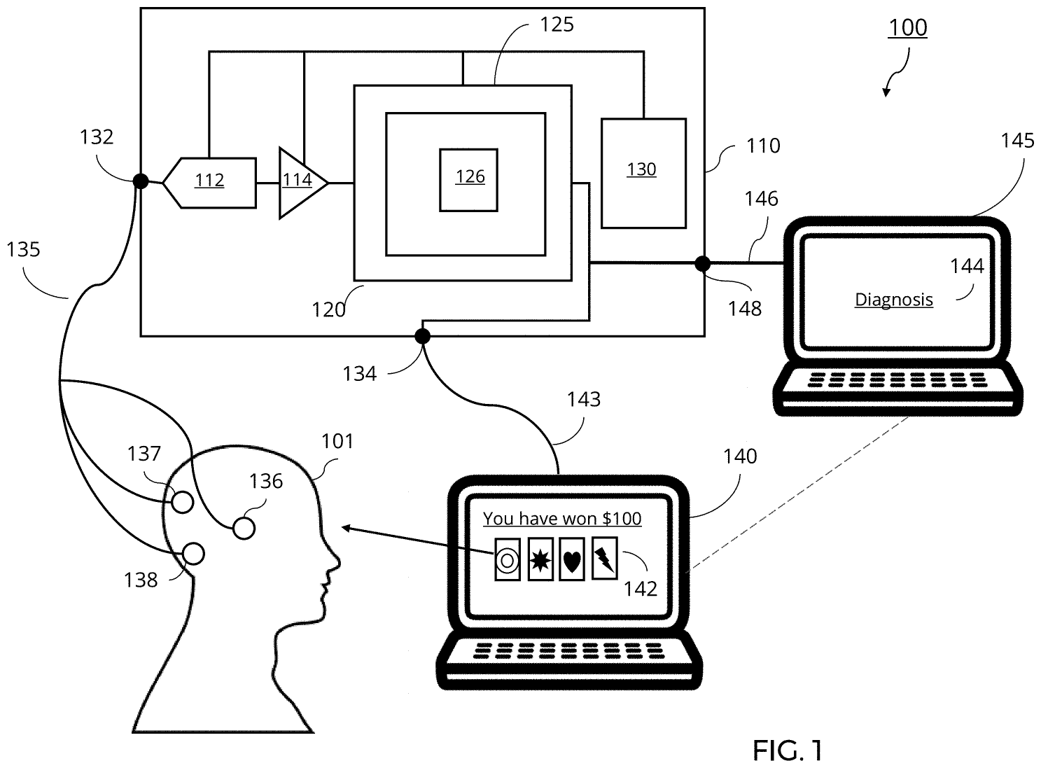

Referring to FIG. 1A, an EEG system 100 features a portable bioamplifier 110 that collects and analyzes EEG signals from a human participant 101 using electrode sensors 136, 137, and 138 attached to participant 101's scalp. Bioamplifier 110 is in communication with a user interface 140 (e.g., a computer with a display) through which the participant interacts with an application 142 to participant 101. Bioamplifier 110 synchronously collects EEG signals from participant 101 while the participant interacts interface 140. The EEG system 100 analyzes the EEG signals, interpreting in real-time participant 101's brain activity responsive to an outcome of a participant's interactions with application 142. Based on the analysis, EEG system 100 determines a likelihood that the participant is suffering from Major Depressive Disorder (MDD). EEG system 100 then presents a depression diagnosis 144 to a user of the system, such as a medical professional, via a second user interface 145 (e.g., a second computer with a display).

Bioamplifier 110 includes jacks 132, 134, and 148 for connecting leads 135, 143, and 146 to the electrode sensors, user interface 140, and user interface 145, respectively. Bioamplifier 110 further includes an analogue-to-digital converter 112, an amplifier 114, and a processing module 120. Although depicted as a single analogue-to-digital converter and a single amplifier, analogue-to-digital converter 112 and amplifier 114 may each have multiple channels, capable of converting and amplifying each EEG signal separately. A power source 130 (e.g., a battery, a solar panel, a receiver for wireless power transmission) is also contained in bioamplifier 110 and is electrically connected to ADC 112, amplifier 114, and processing module 120. In general, analogue-to-digital converter 112 and amplifier 114 are selected to yield digital signals of sufficient amplitude to be processed using processing module 120.

Processing module 120 includes one or more computer processors 125 programmed to analyze and clean amplified EEG signals received from amplifier 114 in real time and to provide a depression diagnosis 144 using a depression diagnosis machine learning (ML) model 126. The computer processors can include commercially-available processors (e.g., a raspberry pi micro-controller) and/or custom components. In some embodiments, processing module 120 includes one or more processors custom designed for neural network computations (e.g., Tensor Processing Unit from Google, Intel Nervanna NNP from Intel Corp. or a Graphics Processing Unit (GPU) from NVIDIA). Generally, processing module 120 should include sufficient computing power to enable real time cleaning and analysis (e.g., neural network inferences) of the EEG signals.

In certain embodiments, bioamplifier 110 is a high-impedance, low-gain amplifier with a high dynamic range. The bioamplifier impedance may be, for example, higher than 10 megaohms (e.g., 12 M\hat{ } or more, 15 M\hat{ } or more, 20 M\hat{ } or more) with a maximum gain of 24× amplification. The dynamic range of bioamplifier 110 should be sufficient to acquire the entire voltage range of typical EEG signals (e.g., 0.1 to 200 μV over frequency ranges of 1 to 100 Hz). As a portable unit, bioamplifier 110 is housed within a compact, robust casing, providing a package that can be readily carried by participant 101, sufficiently robust to remain functional in non-laboratory settings.

In general, electrode sensors 136, 137, and 138 may be any suitable sensor for obtaining EEG signals. For example, sensors 136, 137, and 138 can be dry sensors or may be placed in contact with the participant's scalp using a gel. The sensors can be secured in place using, for example, adhesive tape, a headband, or some other headwear. Typically, one of sensors 136, 137, and 138 is an active sensor.

Generally, the active sensor is positioned to detect brain activity related to the participant's neural reward system. The neural reward system (or simply “reward system”) is a group of neural structures responsible for incentive salience(i.e., motivation and “wanting”, desire, or craving for a reward), associative learning (primarily positive reinforcement and classical conditioning), and positive emotions, particularly ones which involve pleasure as a core component (e.g., joy, euphoria and ecstasy). Reward is the attractive and motivational property of a stimulus that induces appetitive behavior and consummatory behavior, it is believed that the brain structures that compose the reward system are located primarily within the cortico-basal ganglia-thalamo-cortical loop and is primarily dopaminergic in nature.

In some embodiments, the active sensor or active sensors are positioned according to the 10-20 system. For example, an active sensor can be positioned at a Pre-frontal (Fp) position, a Frontal (F) position, a Temporal (T) position, a Parietal (P) position, and/or a Central (C) position and/or at an intermediate position between standard positions (e.g., at an AF, FC, FT, CP, TP, and/or PO position). Furthermore, one or more sensors can be positioned at a z-axis site, according to the 10-20 system (e.g., Pz, FCz). In some implementations, the active sensor is placed at the back of the participant's head, at or close to the participant's inion.

Another one of the sensors is a reference sensor. The EEG signal corresponds to measured electrical potential differences between an active sensor and a reference sensor. The third sensor is a ground sensor. Typically, the ground sensor is used for common mode rejection and can reduce (e.g., prevent) noise due to certain external sources, such as power line noise. In some implementations, the ground and/or reference sensors are located behind the participant's ears, on the participant's mastoid process.

Application 142 presents information to participant 101 that is known to stimulate a person's reward system. For example, in some embodiments, this information relates to a task where the participant can win or lose money, in which case wins and losses elicit discriminable differences in the EEG as a result of the separate functioning of the reward system for these two cases (i.e., winning money is more rewarding than losing money. Reward tasks can be designed to provide rewards such as money, sweet foods, pleasant sounds, or smiling faces.

The application prompts participant 101 to complete the reward task while monitoring EEG signals from the participant. In particular, the EEG system receives the participant's EEG signals in response to the outcome of the reward task and processes the participant's EEG signal response to determine whether the participant suffers from depression as described in more detail below.

In some embodiments, the reward task is a version of the “Doors” task (Hajcak, 2012). In the Doors task, each participant is presented with 2 cartoon doors on a computer screen and is asked to guess which door has a prize behind it. Based on their guess, the participant can either win or lose real money (all participants end up winning the same amount of money across trials). The EEG is marked at the moment when the participant receives feedback about whether they won or lost money; event-related potentials (ERPs) are then formed for “win” and “loss” trials and these ERPs form the basis for the depression diagnosis.

In other embodiments, the reward task is a more explicit conditioning task where participants learn to associate particular visual images with positive outcomes. For example, a participant may learn that a sine grating presented with the sinusoids oriented towards “north” will result in a pleasant tone, while sinusoids oriented towards “east” will result in an unpleasant burst of white noise. The EEG is marked at the moment when the sine gratings are presented; ERPs are then formed for “pleasant” and “unpleasant” unconditioned stimuli and, again, these form the basis for the depression diagnosis.

In other embodiments, the rewarding stimulus is more abstract. It may consist of an emotionally pleasant stimulus, like a cute puppy or a smiling baby. The emotionally pleasant stimulus may be presented for a consciously detectable amount of time (e.g., 5 seconds), for a subliminal amount of time (e.g., 30 milliseconds), or may be flickered repeatedly near alpha rhythm (e.g., at 10 Hz).

Alternatively, or additionally, in certain embodiments, the reward task is a “Doors” task, in which the participant is presented with two adjacent doors and asked to select one of them. After the participant selects a door, the system reveals whether the participant has won or lost, e.g., an amount money. The system measures the participant's brain activity before and after the system reveals the result of the participant's selection.

Techniques for collecting, processing, and determining the participant's choices from EEG signals are described in U.S. patent application Ser. No. 15/855,845 filed Dec. 27, 2017, the entire contents of which are incorporated herein by reference. The EEG system 100 analyzes EEG signals of the participant corresponding to the participant's reaction to the outcome resulting from the participant's choices within the reward task application.

In certain embodiments, application 142 presents information unrelated to reward that are also known to probe systems that are dysfunctional in depression, such as the emotional processing system. For instance, application 142 can present participant 101 with words and/or images associated with a person's emotions. Application 142 can prompt participant 101 by asking the participant to look at graphical images indicate how that image makes them feel. 142B. The images may include images that portray distinct emotions such as “happy,” “sad,” or “neutral.” EEG system 100 measures the participant's EEG activity in response to selecting a particular graphical representation.

Alternatively, or additionally, application 142 can prompt participant 101 by asking the participant to judge whether positive or negative adjectives describe them (e.g., positive: “friendly,” negative, “lonely”). The EEG system 100 measures the participant's EEG activity in response to answering whether a word describes them or not.

In some embodiments, bioamplifier 110 collects EEG signals from participant 101 while the participant is being presented with no stimulus or information via interface 140. For example, bioamplifier 110 can collect resting state EEG signals from participant 101 in order to establish a baseline for the participant against which other EEG signals from the participant can be compared, or for classification.

During operation, processing module 120 receives EEG signals from amplifier 114 and performs pre-processing before providing the signals to the ML depression diagnosis model 126. Pre-processing EEG signals may be referred to as cleaning. Cleaning an EEG signal includes various operations that improve the usability of the signal for subsequent analysis, e.g., by reducing noise in the EEG signal. For example, cleaning the EEG signal can include filtering the signal by applying a transfer function to input data, e.g., to attenuate some frequencies in the data and leave others behind. Processing module 120 may perform basic bandpass filtering on the frequencies that are relevant for the EEG signals, e.g., 0.5 Hz to 20 Hz. Cleaning can also include operations to improve signal quality besides removal of undesirable frequencies. For instance, cleaning can include removing blinks, which digital filtering alone does not do. Cleaning can include performing basic linear de-trending (e.g., i.e. removal of trending in signals) on the raw EEG signals to remove some variability due to skin and sweat. Other signal cleaning operations are also possible. For example, signals can be cleaned using a neural network.

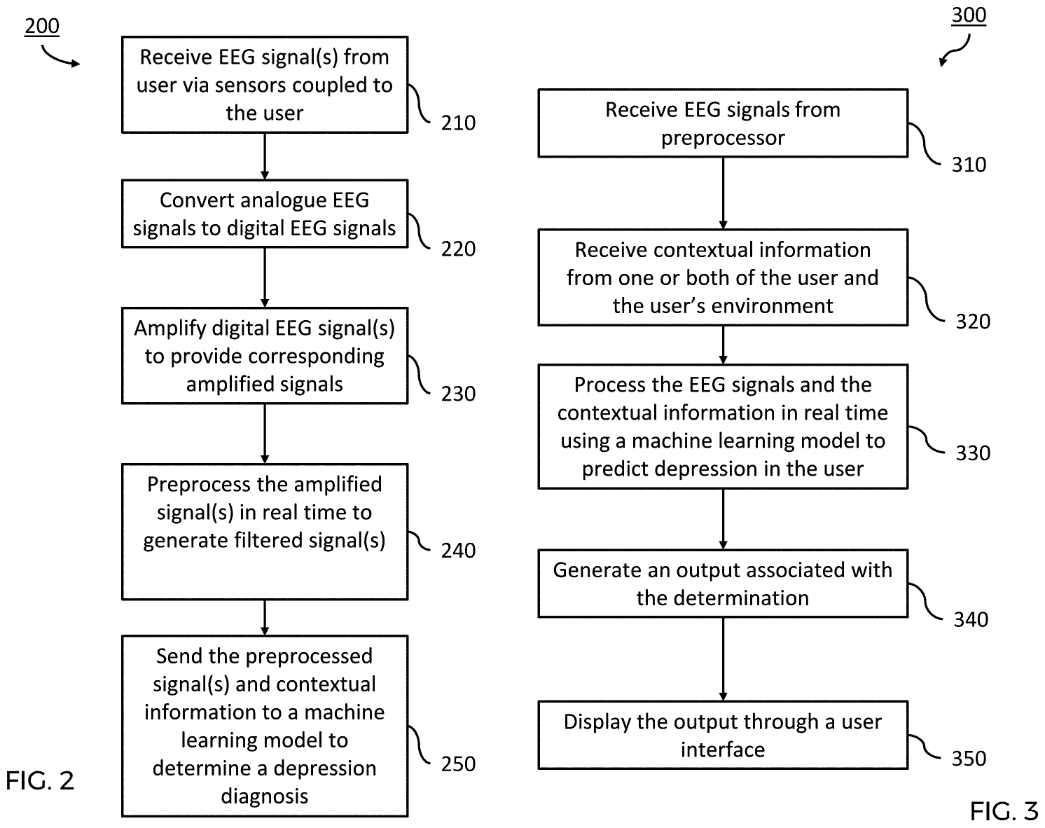

Referring to FIG. 2, the process of digitizing, amplifying, and cleaning an EEG signal is shown in a flowchart 200. An EEG signal, e.g., a time-varying voltage differential between a voltage measured using an active sensor and a reference sensor, is received by a bioamplifier (e.g., bioamplifier 110) from the sensors attached to the participant's scalp (step 210). The frequency at which the sensor voltage is sampled should be sufficient to capture voltage variations indicative of the brain activity of interest (e.g., between 0.1 and 10 Hz, at 10 Hz or more, at 50 Hz or more, at 100 Hz or more). An ADC (e.g., ADC 112) converts the signal from an analogue signal to a digital signal (step 220) and sends the digital signal to an amplifier (e.g., amplifier 114). The digital EEG signal is then amplified (e.g., by amplifier 114) (step 230), and the amplified signal sent to a processor (e.g., processing module 120). The processor (e.g., processing module 120), in real time, preprocesses or cleans the amplified signal (step 240), producing a cleaned EEG signal. The processor (e.g., processing module 120) sends the preprocessed signals and contextual information from the application that prompts a participant for EEG signal responses to a ML model to determine a depression diagnosis (step 250).

As noted previously, a processor (e.g., processing module 120) includes a machine learning depression diagnosis model (e.g., ML depression diagnosis model 126) for analyzing lightly pre-processed EEG signals. In some embodiments, the depression diagnosis model interprets a response of a participant (e.g., participant 101) to information (e.g., information 142) presented via a computer (e.g., computer 140). For example, the computer may present the participant with a reward task in which the participant takes action and either wins or loses. The computer may also present the participant with graphical or word representations of emotions and ask the participant to choose a representation for the participant's current mood.

The ML depression diagnosis model takes in contextual information and the participant's EEG signals at the time the participant is presented with the outcome of the reward task, e.g., when the participant is told whether he or she won or lost, or when the participant provides a mood rating. In other embodiments, the depression diagnosis model interprets a participant's resting state EEG signals, e.g., captured while the participant is at rest and their eyes are closed. The depression diagnosis model associates participants' response to information (e.g., information 142) with contextual information e.g., the context of the participant's response. The system can then identify satisfaction (e.g., acceptance of an option or happiness in an outcome) of the participant in the presented information or in the participant's current state, or dissatisfaction (e.g., rejection of an option or unhappiness in an outcome) in the presented information or in the participant's current state, by classifying the difference between EEG signals. Subject signals are compared to what the model classifies as “depressed” or “non-depressed,” learned from labeled training data, or some other finer grained labeling, in order to determine whether a participant is depressed or not.

Referring to FIG. 3, a process of using a depression diagnosis ML model, e.g., depression diagnosis ML model 126, to diagnose depression is shown in flowchart 300. EEG signals are received from the sensors coupled to a participant (310). The system also receives contextual information from the participant and/or the participant's current environment as described above (step 320). The system provides both the EEG signals and the contextual information to the depression diagnosis ML model as input.

Inputting the EEG signals and contextual information into the ML model can involve extracting values for parameters from the EEG signals and contextual information and inputting these values into the ML model. For example, values for a change in signal amplitude over specific time periods can be extracted from the EEG signals. The time periods can correspond to certain time intervals before, concurrent to, and/or after presenting information to the participant. Where the participant is presented with a reward task, values of the EEG signal within a certain time period (e.g., within 1 second or less, 500 ms or less, 200 ms or less, 100 ms or less) of informing the participant of the outcome (win/lose) can be extracted from the signals and used as input to the ML model. More complex features of the EEG can also be extracted and fed to the ML model. As examples, frequency domain or time x frequency domain information can be submitted to the model, instead of or in addition to, raw time domain information. At the next level of complexity, mathematical operations can be performed on the time, frequency, or time x frequency data before it is fed to the ML model. These include pointwise regression, subtraction or concatenation.

The contextual information can include, for example, a value associated with the outcome of the reward task, a value associated with the mood induced in the participant by an image (e.g., happy, sad, neutral), the intensity of that mood (e.g., very happy vs. moderately happy), a value associated with which image or word a participant was looking at, a value associated with the participant's decision about whether a word applies to them or not, a value associated with the moment the participant closes their eyes to begin providing resting state and another value associated with the moment the participant opens their eyes again.

The model then processes the received input using a machine learning algorithm to diagnosedepression in the participant (step 330). In some embodiments, the machine learning algorithm diagnosesdepression by comparing patterns among labeled EEG signals and contextual information on which the algorithm has been trained with input the algorithm receives to determine how likely the input is to come from the distributions of healthy and depressed data it has seen in the past. The machine learning algorithm selects a diagnosis by determining whether the numerical properties of the EEG are statistically more likely to have come from the trained depressed or non-depressed distribution. For example, the machine learning algorithm may determine that the features of the EEG signal provided by a new patient are more likely to come from a depressed person than a non-depressed person, on the basis of the distribution of those features in labeled examples it has seen in the past. The diagnosis of the ML system is then simply the label of the distribution the input data are statistically most likely to arise from.

In some embodiments, the machine learning algorithm records several outcome reactions from the participant and determines whether, based on reward-related neuro-electric activity, the participant shows significantly different responses between wins and losses. In some examples, a difference is considered significant for diagnosis purposes if it is above a threshold difference value. The threshold difference value can be a learned parameter of the machine learning algorithm. If the participant's EEG signals do not show significantly different reward-related positivity between positive outcomes and negative outcomes, the system can determine that the participant is depressed. If the participant's EEG signals do show different reward-related positivity between positive and negative outcomes, the system can determine that the participant is not depressed. Generally, the difference in reward-related positivity differentiating a healthy participant from a depressed participant can vary depending on the participant and individual responses can vary widely. In some embodiments, a grand mean difference between win/loss outcomes can be about 2.5 micro-Volts or less in depressed participants compared to healthy participants. The ML model can be trained to account for such variation. For example, the ML model can learn to be resilient/robust to such variation through exposure to samples from such data.

The ML model then outputs results indicative of whether the participant's response to the information (step 340) determines that the participant is suffering from major depression. For example, the output can include an indication about whether the participant is presently depressed. The indication can be a binary (e.g., “depressed” or “not depressed”) or can be a statistical likelihood (e.g., a percentage that the participant is depressed).

After diagnosing the participant, the system generates an output associated with the determination (step 340) and displays the output through a user interface (step 350), e.g., on a device for a medical professional. In some embodiments, the bioamplifier can relay the results of depression diagnosis model analysis to another device, which may take certain actions depending on the results. The other device can notify one or more other people (e.g., medical professionals) about the diagnosis via an electronic message service and/or update a database or other electronic record with the diagnosis information. For example, with reference again to the system shown in FIG. 1, the depression diagnosis output from the ML depression diagnosis model 126 can be returned to user interface 140 via lead 143 and/or to user interface 145 via lead 146. In some embodiments, system 100 can be connected to a larger network (e.g., wired and/or wireless network) and the depression diagnosis is returned via a remote, networked user interface.

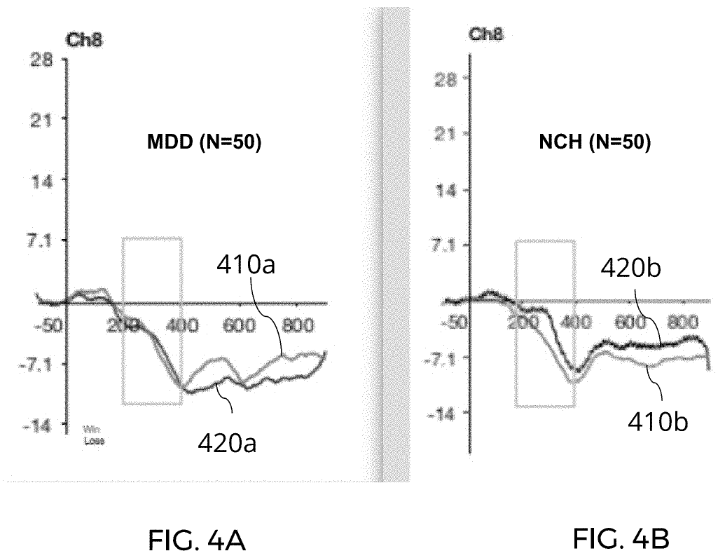

Turning now to illustrative data, FIGS. 4A and 4B show plots of EEG signals for depressed and healthy participants who participated in a reward task. In each case, the plots compare an ERP elicited when the participant wins and an ERP elicited when the participant loses. The x-axis in each plot corresponds to the time, with the origin corresponding to the instant when the participant is informed of the outcome of the reward task. The y-axis shows the relative amplitude of the EEG signal. In each case, the data is averaged for 50 participants (N=50) from each category of participants.

In FIG. 4A , the plot shows EEG signals of the depressed participants. Notably, for the time period from 200 ms to 400 ms, signal 410a (corresponding to a win in the reward task) substantially overlaps with signal 420a (corresponding to a loss in the reward task). For example, to a depressed person, wins and losses do not produce a significant difference in reward-related positivity. Therefore, a depressed person can feel similarly in response to both scenarios, whether the outcome is a win or a loss, which is reflected in the EEG signals from 200 ms to 400 ms in FIG. 4A.

In FIG. 4B, the plots shows EEG signals from healthy (i.e., not depressed) participants. In contrast to FIG. 4A, the signal 410b for a win and the signal 420b for a loss for a healthy person shows a greater separation for the time period between 200 ms and 400 ms (identified by the grey box in FIGS. 4A and 4B). It is believed this plot shows that, for a healthy person, wins and losses produce a noticeable difference in reward-related positivity.

Accordingly, in some cases EEG signals representing a participant's response to wins will only appear to show an increase in reward-related positivity—compared to losses—to the extent that the participant is not depressed. Thus, a ML model can associate differences in reward-related positivity between outcome signals for a participant or between the signals of the participant and the signals from training participants with either a depressed or healthy outlook and return a depression diagnosis to a computer.

In general, a variety of ML models can be used to perform the depression diagnosis. In some embodiments, the ML depression diagnosis model is a neural network which employs one or more layers of nonlinear units to determine whether a participant is depressed given a sequence of EEG data. A variety of neural networks can be used to analyze and classify data. For example, the neural network can be a convolutional neural network model, a recurrent neural network, a generative adversarial network model, or an autoencoder. In some implementations, lower dimensional models, e.g., a multilayer perceptron or autoencoder can be implemented. The minimum number of features that can be used to achieve acceptable accuracy in decoding the participant's depression is preferred for computational simplicity. Optimized models may be trained or simulated in constrained computing environments in order to maximize speed, power, or interpretability. Three primary features of optimization are (1) the number of features extracted, (2) the “depth” (number of hidden layers) of the model, and (3) whether the model implements recurrence. These features are balanced in order to achieve the highest possible accuracy while still allowing the system to operate in near real time on the embedded hardware.

The neural network may be a deep neural network that includes two or more hidden layers in addition to the input and output layers. The output of each hidden layer is used as input to another layer in the network, i.e., another hidden layer, the output layer, or both. Some layers of the neural network may generate an output from a received input, while some layers do not, e.g., the layers remain “hidden.” The network may be recurrent or feedforward. It may have a single output or an ensemble of outputs, it may have an ensemble of architectures with a single output or a single architecture with a single output. In some embodiments, the ML depression diagnosis model may have added convolutional layers that represent filters so that the EEG signals can be cleaned and processed by the same ML model.

In some embodiments, the ML model uses subselection in which the model only compares the current participant's brain activity with other participant samples that are most similar to that of the participant in order to determine a depression diagnosis. Similarity to other participants can be operationalized with standard techniques such as waveform convolution and normalized cross correlation. Alternatively, the machine learning model compares the participant's brain activity to that of all brain activity present in a large dataset (e.g., in an embodiment that employs a nearest-neighbor-type of model). The dataset may contain labeled brain activity samples from one or more other participants. Samples for comparison are drawn either from (1) a data system's internal participant data or (2) data collected from external participants who have opted-in to having their data be included in the comparison database. All samples are anonymized and are non-identifiable.

In general, ML models for depression diagnosis can be trained on EEG data in order to examine patterns of brain activity and generate a depression diagnosis. Once the model is trained broadly across multiple situations, outcomes, and people, the system can use the ML model on any person for a depression diagnosis without further training. In some embodiments, the more similar the new situation or outcome is to a trained situation or outcome, the more effective this transfer will be. However, in certain embodiments, ML models can return accurate results based on novel data on which it has not been trained.

To train the ML depression diagnosis model the system provides the model with: (1) labeled EEG signals from multiple people and (2) context about participants' environments, e.g., what is being displayed to the participant while each labeled signal was collected or whether a particular participant is in a resting state during collection of the labeled signal.

In some embodiments, the ML model can be non-neural network based models including, but not limited to a support vector machine, a random forest/decision tree ensemble.

In some embodiments, the EEG signal data is labeled by at least one expert to distinguish between depressed and non-depressed EEG signals and classify the data as either data that corresponds to a depressed person or a healthy person. Contextual information may include information about the participant, the participant's current environment, information that is currently being displayed to the participant from a computer application, e.g., a reward task, and/or other useful information that can be used to determine whether the participant is depressed, e.g., where the participant is located, the temperature of the participant's surroundings, the availability of computing devices, information from wearables, information from cameras, information from smart home devices, information from computing devices, information about the state of calibration of the bioamplifier, the current time, and the current weather. The machine learning model may map the EEG signals in a time series to a depression diagnosis using the contextual information

The EEG system performs multiple nonlinear operations on data to train the model using the labeled data and the context that corresponds to the labeled data as input. Essentially, the training is supervised machine learning. In some implementations as discussed above, the ML model may be pre-trained on representative information from test participants. The ML model may then not need to be trained on each new participant. In other implementations, the ML model is trained on each participant individually.

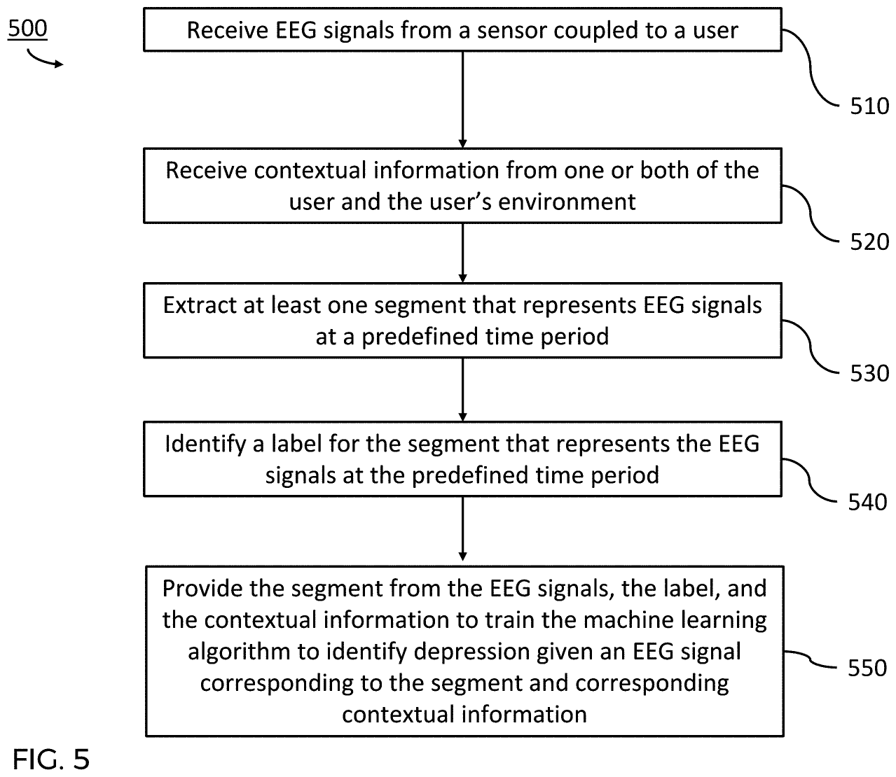

Referring to FIG. 5, a process for training a depression diagnosis machine learning model using collected EEG signals is shown in flowchart 500. EEG signals are received from sensors coupled to training participants (step 510). The signals represent time-varying voltage differentials between voltage measured using an active sensor and a reference sensor. The system also receives contextual information, which identifies circumstances that form the setting for the participant's specific EEG signals (step 520). This contextual information can come from the participant or other computing devices in the participant's environment. The system then extracts segments, forming limited time series, from the EEG signals representing neural activity from a predefined time period just prior to the moment when the participant is shown an outcome or asked about the participant's emotional state to a time period after when the participant has registered an emotional response. For example, when the system displays the outcome of a reward task to the participant, the EEG system records EEG signals from the participant from a time prior to the outcome being shown to the participant and to a defined time after the outcome being shown to determine the participant's response to the outcome (step 530). ML model can classify each response as either “depressed” or “healthy,” as discussed above. The EEG system can then identify the classifications using labels for the data (step 540). The system provides the depression diagnosis ML model with time series of EEG signals that represent neural activity corresponding to a certain scenario, the label applied to the EEG signals, and the contextual information related to the EEG signals (step 550) to train the model on specific depression data to be able to recognize depression in other participants.

In some embodiments, the system does not prompt the participant for an emotional response, but instead records EEG signals as the participant is in a resting state. The contextual information for these embodiments will include information about the participant and/or the participant's environment in a resting state.

While the systems described above both feature a portable bioamplifier (i.e., bioamplifier 110), that connects with either a computer or other interface, other implementations are also possible. For example, the components of a bioamplifier (e.g., bioamplifier 110) can be integrated into another device, such as a mobile phone or tablet computer. Moreover, while the foregoing systems includes sensors that are connected to the portable bioamplifier using leads, other connections, e.g., wireless connections, are also possible.

In general, the EEG systems described above can use a variety of different sensors to obtain the EEG signals. In some implementations, the sensor electrodes are “dry” sensor which feature one or more electrodes that directly contact the participant's scalp without a conductive gel. Dry sensors can be desirable because they are simpler to attach and their removal does not involve the need to clean up excess gel. A sensor generally includes one or more electrodes for contacting the participant's scalp.

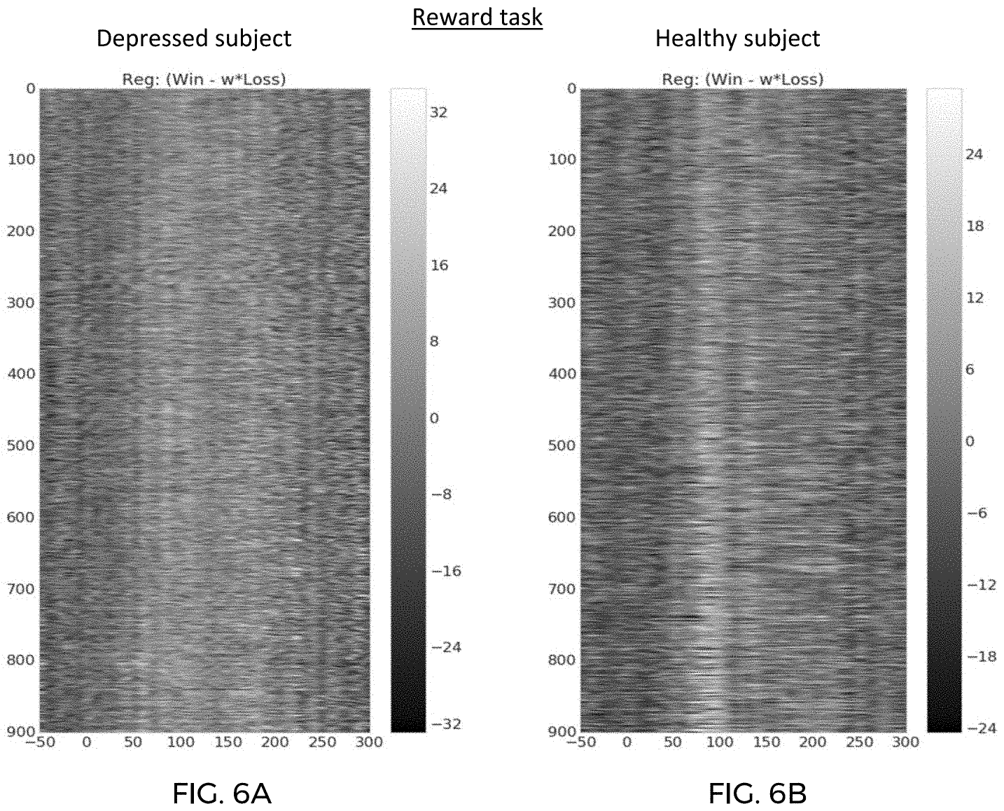

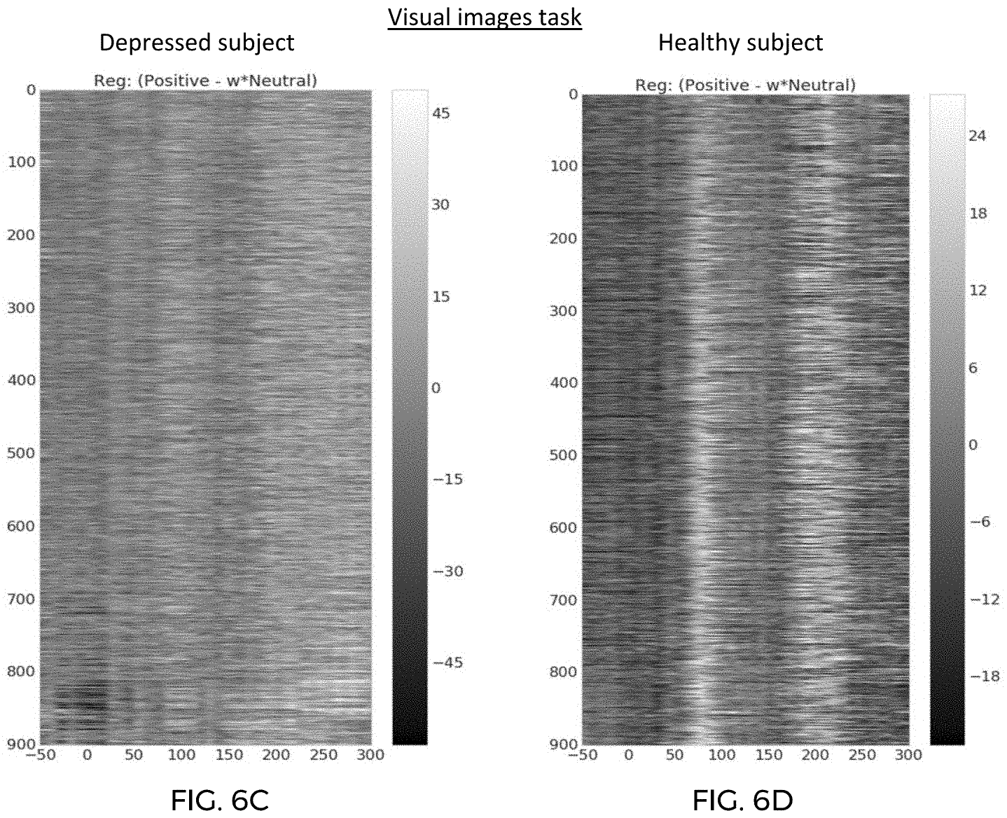

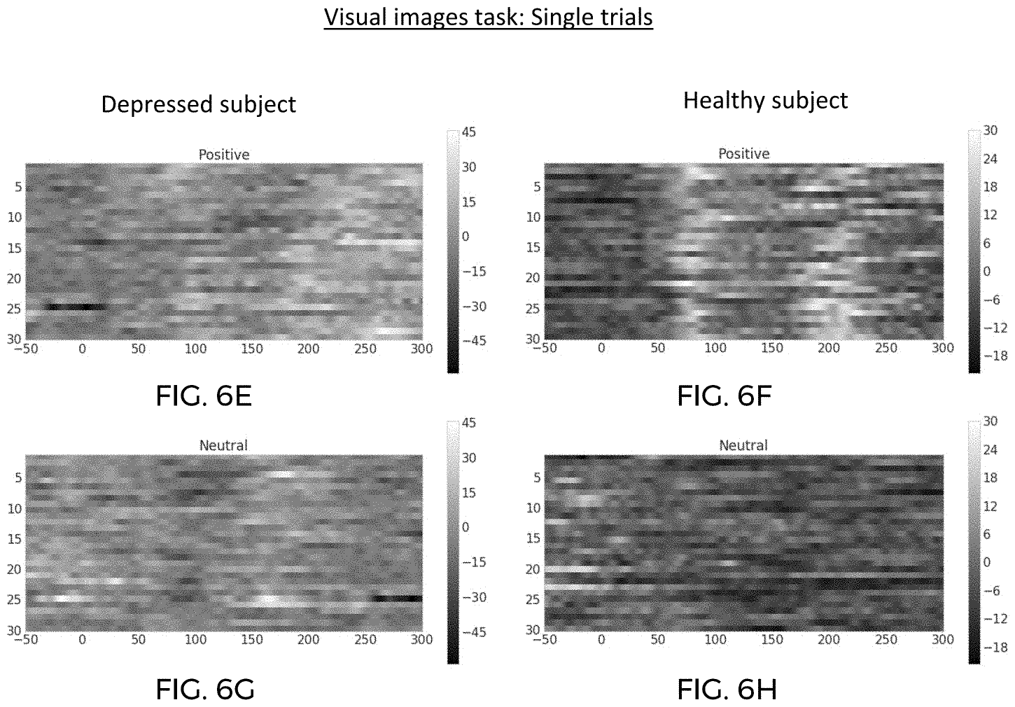

Referring to FIGS. 6A-6D, results of studies performed using the above-described techniques are illustrative for demonstrating its ability to distinguish a healthy participant from a depressed participant. The plots shown in these figures contrast an EEG response from a depressed participant (FIGS. 6A and 6C) with that of a healthy participant (FIGS. 6B and 6D). The plots in FIGS. 6A and 6B were generated from a study in which the depressed participant and the healthy participant each completed 60 reward tasks in which 30 resulted in a “win” and 30 resulting in a “loss.” The plots in FIGS. 6C and 6D were generated from a study in which the depressed participant and the healthy participant each completed visual image tasks in which “positive” and “neutral” images were presented to the participants. FIGS. 6E-6H are plots that illustrate individual EEG trials used to generate the plots in FIGS. 6C and 6D.

In FIGS. 6A and 6B, the x-axes in each plot represents time in seconds (units are amplifier samples at 250 Hz). The x-axis of each plot shows a time from 50 ms prior to presenting the participant with the result of a reward task until 200 ms after the result is shown. The y-axis of each plot is an ordinal axis, representing a stack of all 900 win/loss combinations obtained by comparing each of 30 wins with each of 30 losses. The color of the each pixel represents the value of the EEG at that time. More specifically, the intensity of each pixel represents the magnitude of the EEG value Generally, a variety of possible distance functions can be used to compare such data. For instance, the distance function may simply be a difference in EEG voltage between each “win” EEG and each “loss” EEG. More complex functions, such as regression or logistic regression, can also be used.

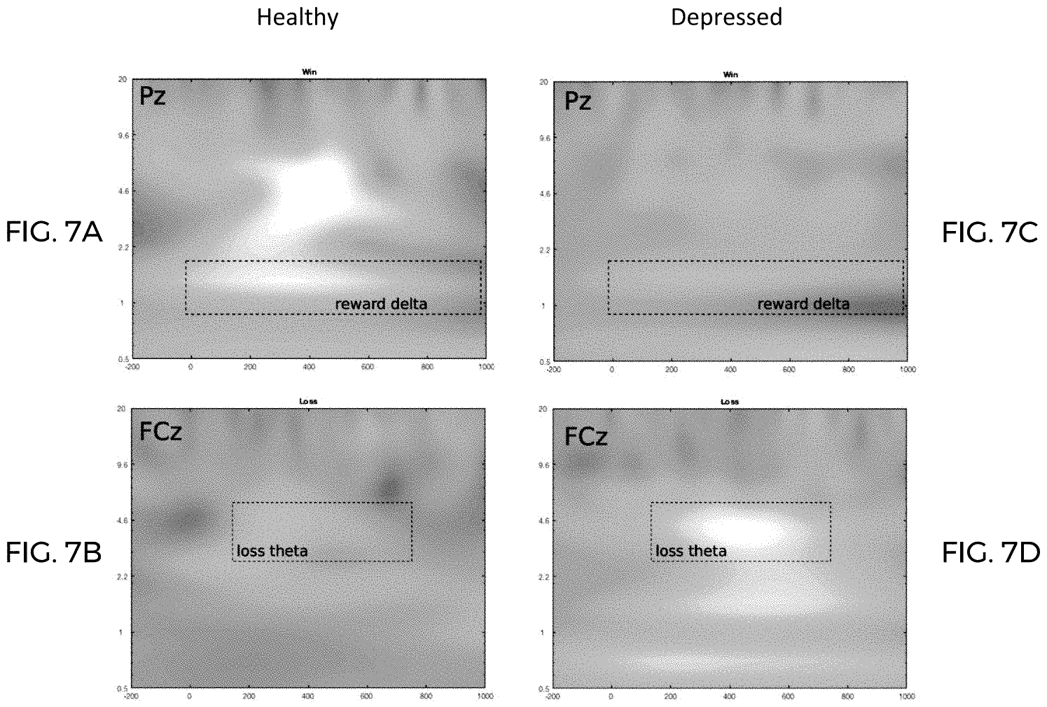

Other graphical visualizations of the EEG data can be used. For example referring to FIGS. 7A-7D, it may be instructive to visualize the time-varying frequency response of EEG data. For example, FIGS. 7A-7D show heat maps of EEG activity in which the x-axes represent time (in ms) from 200 ms prior to presenting the participant with the result of a reward task until 1,000 ms after the result is shown. The y-axes represent the frequency of the EEG response, from 0.5 Hz to 20 Hz. The shading in the heat map represents the signal strength at each frequency. Darker shades represent low signal strength and lighter shades represent high signal strength.

The plots illustrate the difference in activity between a healthy participant and a depressed participant. Specifically, FIGS. 7A and 7B show the brain activity for a healthy participant and FIGS. 7C and 7D show brain activity for a depressed participant. FIGS. 7A and 7C show the EEG spectrogram for a “win” scenario of the reward task, and FIGS. 7B and 7D show the spectrogram for a “loss.” For each participant, brain activity was measured with a sensor in the Pz position for the “win” scenario and in the FCz position for the “loss” scenario. In FIGS. 7A and 7C, the “reward delta” area corresponds to a frequency range of interest in which an EEG signal is sensitive to a reward (e.g., from a “win”). Conversely, in FIGS. 7B and 7D, the area identified as “loss theta” corresponds to a frequency range in which an EEG signal is known to be sensitive to a loss.

Accordingly, a healthy participant demonstrate a strong response to a “win” in the reward delta area (FIG. 7A), but a muted response to a “loss” in the loss theta (FIG. 7C). Conversely, a depressed participant demonstrates a muted response to a “win” in the reward delta (FIG. 7C), but a strong response to a “loss” in the loss theta (FIG. 7D).

In some embodiments, visual representations of the analyzed EEG data can be presented to a user of the system (e.g., a medical professional or technician), such as that shown in FIGS. 6A-6B and/or FIGS. 7A-7D, to facilitate diagnosis. In some embodiments, the visualizations can include the EEG waveforms, as illustrated in FIG. 3. Any way of visualizing EEG signal data as a comparison among a participant and healthy and/or depressed people can be contemplated for the user interface 145.

Embodiments of the participant matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the participant matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively, or in addition, the program instructions can be encoded on an artificially-generated propagated signal, e.g., a machine-generated electrical, optical, or electromagnetic signal, which is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user and/or a participant, embodiments of the participant matter described in this specification can be implemented on a computer having a display device, e.g., a CRT (cathode ray tube) or LCD (liquid crystal display) monitor, for displaying information to the user and/or participant and a keyboard and a pointing device, e.g., a mouse or a trackball, by which the user and/or participant can provide input to the computer. Other kinds of devices can be used to provide for interaction with a user and/or participant as well; for example, feedback provided to the user and/or participant can be any form of sensory feedback, e.g., visual feedback, auditory feedback, or tactile feedback; and input from the user and/or participant can be received in any form, including acoustic, speech, or tactile input. In addition, a computer can interact with a user and/or participant by sending documents to and receiving documents from a device that is used by the user and/or participant; for example, by sending web pages to a web browser on a user and/or participant device in response to requests received from the web browser. Also, a computer can interact with a user and/or participant by sending text messages or other forms of message to a personal device, e.g., a smartphone, running a messaging application, and receiving responsive messages from the user and/or participant in return.

Embodiments of the participant matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user and/or participant can interact with an implementation of the participant matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML, page, to a user device, e.g., for purposes of displaying data to and receiving user input from a user and/or participant interacting with the device, which acts as a client. Data generated at the device, e.g., a result of the user and/or participant interaction, can be received at the server from the device.

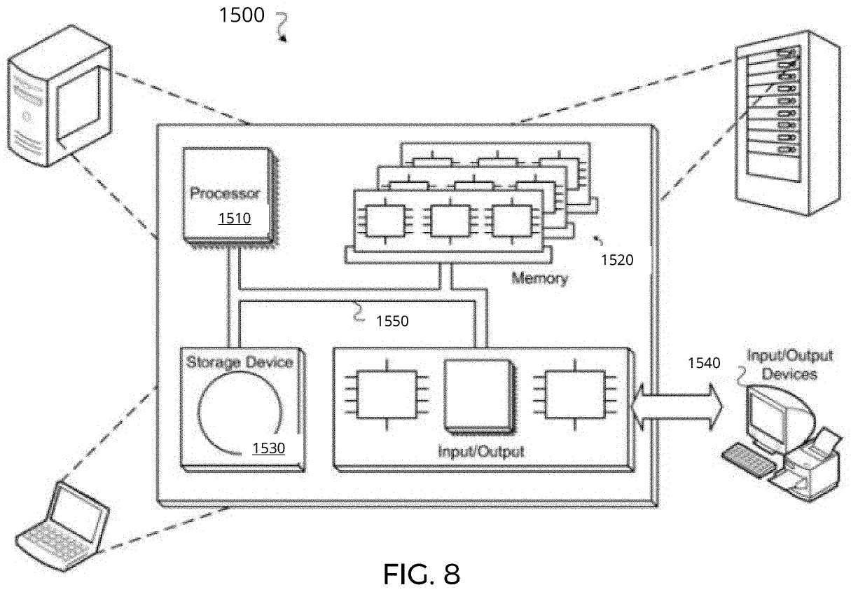

An example of one such type of computer is shown in FIG. 8, which shows a schematic diagram of a generic computer system 1500. The system 1500 can be used for the operations described in association with any of the computer-implemented methods described previously, according to one implementation. The system 1500 includes a processor 1510, a memory 1520, a storage device 1530, and an input/output device 1240. Each of the components 1510, 1520, 1530, and 1540 are interconnected using a system bus 1550. The processor 1510 is capable of processing instructions for execution within the system 1500. In one implementation, the processor 1510 is a single-threaded processor. In another implementation, the processor 1510 is a multi-threaded processor. The processor 1510 is capable of processing instructions stored in the memory 1520 or on the storage device 1530 to display graphical information for a user interface on the input/output device 1540.

The memory 1520 stores information within the system 1200. In one implementation, the memory 1520 is a computer-readable medium. In one implementation, the memory 1520 is a volatile memory unit. In another implementation, the memory 1520 is a non-volatile memory unit.

The storage device 1530 is capable of providing mass storage for the system 1500. In one implementation, the storage device 1530 is a computer-readable medium. In various different implementations, the storage device 1530 may be a floppy disk device, a hard disk device, an optical disk device, or a tape device.

The input/output device 1540 provides input/output operations for the system 1500. In one implementation, the input/output device 1540 includes a keyboard and/or pointing device. In another implementation, the input/output device 1540 includes a display unit for displaying graphical user interfaces.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

As used herein, the term “real-time” refers to transmitting or processing data without intentional delay given the processing limitations of a system, the time required to accurately obtain data and images, and the rate of change of the data and images. In some examples, “real-time” is used to describe concurrently receiving, cleaning, and interpreting EEG signals. Although there may be some actual delays, such delays generally do not prohibit the signals from being cleaned and analyzed within sufficient time such that the data analysis remains relevant to provide decision-making feedback and accomplish computer-based tasks.

Particular embodiments of the participant matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous. Wireless or wired connections may be advantageous for different use cases. Miniaturized components may replace existing components. Other data transmission protocols than those listed may be developed and implemented. The nature of the ML systems used for both data cleaning and classification may change.

What is claimed is:

1. A method for analyzing electroencephalogram (EEG) signals, comprising:
presenting a human participant with information known to stimulate a person's neural reward system;
receiving an EEG signal from a sensor coupled to the human participant in response to presenting the participant with the information, the EEG signal being associated with the participant's neural reward system;
contemporaneously with receiving the EEG signal, receiving contextual information related to the information presented to the human participant;
processing the EEG signal and the contextual information in real time using a machine learning model trained to associate depression in the person with EEG signals associated with the person's neural reward system and the presented information; and
diagnosing whether the participant is experiencing depression based on an output of the machine learning model.

2. The method of claim 1, wherein the information known to stimulate the person's neural reward system is associated with a reward task.

3. The method of claim 2, wherein the reward task comprises two outcomes, a first outcome that corresponds to a win for the participant and a second outcome that corresponds to a loss for the participant; and
wherein the contextual information comprises information about the outcome of the reward task.

4. The method of claim 1, wherein the information known to stimulate a person's neural reward system comprises presenting to the participant a graphical image of two objects, each object concealing an outcome that comprises either a winning outcome or a losing outcome.

5. The method of claim 4, further comprising:
prompting the participant to select one of the two objects,
wherein the contextual information comprises the outcome concealed by the selected one of the objects.

6. The method of claim 1, wherein processing the EEG signals comprises extracting one or more parameters characteristic of the EEG signals for inputting into the machine learning model.

7. The method of claim 6, wherein prior to extracting the one or more parameters, processing the EEG signals comprises filtering the EEG signals.

8. The method of claim 1, wherein in order to determine a depression diagnosis, the machine learning model identifies a strong response in a loss theta region of the EEG signals associated with the person's neural reward system.

9. The method of claim 1, wherein generating an output associated with the determination comprises:
providing, for display on a participant interface, a graphical representation that depicts the participant's EEG signals with that of a healthy individual and a depressed individual.

10. The method of claim 9, wherein the participant interface includes at least one visualization of the participant's EEG signals.

11. The method of claim 10, wherein the visualization can be a waveform of the signal, a heat map, or a time frequency representation.

12. The method of claim 1, wherein the contextual information includes at least one of:
the participant's location, a temperature of the participant's surroundings, an outcome that affects the participant's reward-related positivity, available computing devices, activities occurring on available computing devices, information from wearables, information from cameras, information from smart home devices, a current time, and current weather.

13. The method of claim 1, wherein the machine learning model comprises a neural network or other machine learning architecture.

14. The method of claim 1, wherein the machine learning model has a mapping function that maps a time series or frequency spectrum of values corresponding to EEG signals to a classification based on labeled training data and the contextual information.

15. The method of claim 1, wherein the machine learning model is trained using training data from healthy and depressed individuals and wherein the data is labeled for a depression diagnosis and associated with specific contextual information.

16. The method of claim 1, wherein prior to processing the EEG signals and the contextual information in real time using a machine learning model, pre-processing the EEG signals using bandpass filtering, linear de-trending, and/or another machine learning model.

17. The method of claim 1, further comprising:
prior to displaying the output through a participant interface on a device for a medical professional, sending the generated output to the device medical professional using a cloud service.

18. The method of claim 12, wherein the training data is labeled using depression diagnosis labels prior to training the model on the data.

19. A depression diagnosis system, comprising:
one or more processors;
one or more tangible, non-transitory media operably connectable to the one or more processors and storing instructions that, when executed, cause the one or more processors to perform operations comprising:
presenting a human participant with information known to stimulate a person's neural reward system;
receiving an EEG signal from a sensor coupled to the human participant in response to presenting the participant with the information, the EEG signal being associated with the participant's neural reward system;
contemporaneously with receiving the EEG signal, receiving contextual information related to the information presented to the human participant;
processing the EEG signal and the contextual information in real time using a machine learning model trained to associate depression in the person with EEG signals associated with the person's neural reward system and the presented information; and
diagnosing whether the participant is experiencing depression based on an output of the machine learning model.

20. A non-transitory computer readable storage medium storing instructions that, when executed by at least one processor, cause the at least one processor to perform operations comprising:
presenting a human participant with information known to stimulate a person's neural reward system;
receiving an EEG signal from a sensor coupled to the human participant in response to presenting the participant with the information, the EEG signal being associated with the participant's neural reward system;
contemporaneously with receiving the EEG signal, receiving contextual information related to the information presented to the human participant;
processing the EEG signal and the contextual information in real time using a machine learning model trained to associate depression in the person with EEG signals associated with the person's neural reward system and the presented information; and
diagnosing whether the participant is experiencing depression based on an output of the machine learning model.
